(19) United States
(12) Patent Application Publication
(10) Pub. No.: US 2012/0109276 A1
(43) Pub. Date: May 3, 2012

(54) CATHETER, SYSTEM FOR APPLYING AN INTRALUMINAL ENDOPROSTHESIS AND METHOD FOR PRODUCING SAME

(75) Inventors: Tobias Diener, Fuerth (DE); Elisabeta Burean, Erlangen (DE); Matthias Fringes, Ansbach (DE)

(73) Assignee: BIOTRONIK AG, Buelach (CH)

(21) Appl. No.: 13/277,694

(22) Filed: Oct. 20, 2011

Related U.S. Application Data
(60) Provisional application No. 61/407,478, filed on Oct. 28, 2010.

(51) Int. Cl. A61F 2/84; B23P 11/00; A61M 29/00

(52) U.S. Cl. 623/1.11; 604/103.06; 29/428

(57) ABSTRACT

The present invention relates to a catheter comprising a balloon (10, 30) and at least one pharmaceutically active substance. To enable more accurate dosing of the pharmaceutically active substance, the pharmaceutically active substance is disposed in a reservoir (15, 35) inside the balloon (10, 30) or on the balloon (10, 30), wherein a wall (14, 32, 34) that closes the reservoir toward the outside comprises at least one predetermined breaking point (16). Furthermore, a system for applying an intraluminal endoprosthesis, preferably a stent, in a body cavity, and simple and cost-effective methods for manufacturing the catheter and the system are described.

### CROSS REFERENCE TO RELATED APPLICATIONS

This invention claims benefit of priority to U.S. patent application Ser. No. 61/407,478 filed Oct. 28, 2010; the contents of which are herein incorporated by reference in their entirety.

### TECHNICAL FIELD

The present invention relates to a catheter comprising a balloon and at least one pharmaceutically active substance. The invention furthermore relates to a system for applying an intraluminal endoprosthesis, preferably a stent, in a body cavity, which is composed of the intraluminal endoprosthesis and such a catheter. Moreover, the invention relates to a method for manufacturing a catheter and a method for manufacturing a system for applying an intraluminal endoprosthesis.

### BACKGROUND

Catheters are small tubes or tubes having different diameters, which can be inserted into the particular body cavity to be treated. “Balloon catheters” are used primarily in angioplasty to expand or reopen a vessel. Such a balloon catheter is composed of a tube that includes an initially non-dilated balloon in a specified region along the tube. To perform treatment using a balloon catheter, a guide wire is first inserted into the vessel to be treated. Next, the balloon catheter is advanced along the guide wire to the site of the vessel to be treated, thereby placing the balloon in the region of the site of the vessel to be treated, where a stenosis is located, for instance. The balloon is then dilated, i.e. unfolded and/or expanded, thereby reopening or expanding the site to be treated. Finally, the balloon is deflated and removed from the vessel along the guide wire. At the same time or subsequently, the guide wire is also withdrawn from the vessel. By expanding or reopening the vessel, the flow of the bodily fluid in the vessel is no longer hindered, or is hindered to a lesser extent.

Balloon catheters of that type can also be used to apply intraluminal endoprostheses at a site to be treated in a body cavity.

Intraluminal endoprostheses, in particular in the form of stents, often comprise a tubular or hollow-cylindrical matrix lattice that is open at both longitudinal ends. An endoprosthesis of that type is inserted into the body cavity preferably using a catheter, and is dilated using the balloon disposed on the catheter. Self-expandable stents (e.g. Nitinol stents) are transported with the catheter to the vascular site to be treated, where they are released. Once the catheter is removed, the endoprosthesis is used to support the body cavity. Stents have become established in particular for use to treat vascular diseases. The use of stents enables constricted regions in the vessels to be permanently expanded in such a manner that the lumen is increased.

During and/or after treatment with a catheter and/or a system for applying an intraluminal endoprosthesis, drugs (also referred to below as a pharmaceutically active substance) are administered for supporting the treatment with the catheter or the intraluminal endoprosthesis. These pharmaceutically active substances are intended primarily to prevent inflammation of the tissue or to prevent the body cavity from closing again (restenosis).

Catheters which have a coating of a pharmaceutically active substance on the outside of the balloon are being used and tested at the present time. It is thereby made possible to administer the drug once, thereby treating only the surrounding tissue while the balloon is in the opened state. Moreover, the drug acts only on the surface of the tissue.

The known catheters or systems for applying an intraluminal endoprosthesis often have the problem that a drug coating of the type described above is mechanically unstable. For example, a large quantity of the pharmaceutically active substance becomes lost during implantation by the active ingredient being scraped off or prematurely rinsed out, thereby enabling particles to form which, in the worst case, can cause the treated vessel to close. Furthermore, the mechanical instability of the coating also makes it difficult to dose the active ingredient exactly. Further limiting factors of the previous solutions are the rate of diffusion of the drug into the tissue, or the lipophilic character thereof, and the opening time of the balloon, which likewise result in an inexact dosing of the active ingredient.

### SUMMARY

The object is thus to create a catheter that makes it possible to apply a targeted, exact dosage of a pharmaceutically active substance. A further object is to create a related system of catheter and intraluminal endoprosthesis. In addition, the object is to provide methods for manufacturing a catheter and/or a system comprising a catheter and an intraluminal endoprosthesis, which make it possible to manufacture these objects cost-effectively.

The above-described object is solved using a catheter, in which the pharmaceutically active substance is disposed in a reservoir inside the balloon or on the balloon, wherein a wall that closes the reservoir toward the outside comprises at least one predetermined breaking point.

The at least one predetermined breaking point of the reservoir is provided in the wall of the reservoir, which lies in the direction of the intended administration of the pharmaceutically active substance. The predetermined breaking point is closed during the handling of the catheter outside of the body to be treated and during application of the catheter to the region of the body where the treatment will take place. No pharmaceutically active substance emerges from the reservoir during this time. As soon as the balloon is dilated and the dilation pressure also acts on the reservoir, the predetermined breaking point breaks or tears, and the pharmaceutically active substance is carried outward in the direction of the tissue to be treated since the pharmaceutically active substance is likewise under excessive pressure, in particular due to the dilation pressure and the balloon outer side pressing against the vascular wall or due to the volume of the reservoir, which shrinks during dilation.

The advantage of the catheter according to the invention is that the pharmaceutically active substance can be dosed exactly since the pharmaceutically active substance is not provided as a coating on the surface, but rather is contained in an initially closed reservoir. The active ingredients therefore cannot be mechanically removed from the surface of the balloon during implantation. By opening the predetermined breaking point during dilation of the balloon, the pharmaceutically active substance is “injected” in a targeted manner into the surrounding tissue of the body cavity to be treated. As a result, lipophilic materials and materials having a hydrophilic character can be used as pharmaceutically active substances. The pharmaceutically active substance is released by the dilation pressure (2 to 30 bar, preferably 10 to 18 bar) which is applied to dilate the balloon. The dilation pressure that is applied causes the predetermined breaking point to open at least indirectly (e.g. by tearing or bursting). As a result, the pharmaceutically active substance is conveyed with overpressure out of the reservoir and through the predetermined breaking point, and is injected into the surrounding tissue, thereby making it possible to reach certain depths of the tissue in a controlled manner. It is therefore ensured that the drugs will not be rinsed off the tissue surface after the balloon is deflated.

According to a known outer coating of the balloon with a pharmaceutically active substance, the pharmaceutically active substance was transferred to the affected tissue in a short period of time during which the balloon lines the body cavity to be treated. By contrast, in the case of the catheter according to the invention, the pharmaceutically active substance is injected over a longer period of time, namely during the entire period of time when the dilation pressure is applied to the balloon. By adjusting the period of time during which dilation pressure is applied to the balloon, it is likewise possible to control the quantity of drug that is dispensed into the tissue.

A “pharmaceutically active substance” (or therapeutically active or effective substance) is understood to mean, within the scope of the invention, a plant-based, animal-based, or synthetic active agent (drug) or a hormone that is used in suitable doses as a therapeutic agent to influence states or functions of the body, as a replacement for active agents that are produced naturally by human or animal bodies, such as insulin, and to eliminate or render harmless pathogens, tumors, cancer cells, or foreign substances. The release of the substance into the surroundings of the endoprosthesis has a positive effect on the healing process or counteracts pathological changes in the tissue after a surgical procedure, prevents inflammation, or serves to render diseased cells harmless in oncology.

Pharmaceutically active substances of that type typically have e.g. an anti-inflammatory and/or anti-proliferative and/or spasmolytic effect, thereby making it possible to prevent e.g. restenoses, inflammation, or (vascular) spasms. In particularly preferred embodiments, substances of that type can be composed of one or more substances from the active ingredient group of calcium channel blockers, lipid regulators (e.g. fibrates), immunosuppressants, calcineurin inhibitors (e.g. Tacrolimus), antiphlogistics (e.g. cortisone or dichlofenac), anti-inflammatory drugs (e.g. imidazole), antiallergenics, oligonucleotides (e.g. dODN), estrogens (e.g. genistein), endothelium formers (e.g. fibrin), steroids, proteins, hormones, insulins, cytostatic agents, peptides, vasodilators (e.g. Sartane) and the anti-proliferative substances of taxols or taxanes, preferably in this case paclitaxel or sirolimus, and the class of limus drugs.

In the case of the present invention, one or a large number of pharmaceutically active substances can be disposed in the reservoir. It is also possible to provide one or more of the pharmaceutically active substances in microspheres or microcapsules which induce a delayed (retarded) release of the particular pharmaceutically active substance. In this case, when the predetermined breaking point is opened, the microspheres or microcapsules are released into the tissue to be treated.

A particularly simple embodiment of a related reservoir for a pharmaceutically active substance is obtained by providing the balloon with an inner wall and an outer wall, and disposing the reservoir between the inner wall and the outer wall.

According to another simple design of a reservoir for a pharmaceutically active substance, a pocket wall is used to design a pocket as a reservoir which can be disposed on the inner side or the outer side of a balloon wall. The pocket wall is connected to the balloon wall along its entire circumference using a joining process, preferably welding or bonding. By using such a design of a reservoir for a pharmaceutically active substance, it is made possible in a particularly advantageous manner to subsequently install reservoirs for pharmaceutically active substances in any predefined sites in the region of the balloon. If the pocket is installed on the inner side (luminal side) of the balloon wall, the predetermined breaking point in the balloon wall is disposed in the region of the pocket. If the pocket with the reservoir containing the pharmaceutically active substance is provided on the outer side or the abluminal side of the balloon wall, the at least one predetermined breaking point is disposed in the pocket wall.

The pocket wall and the balloon wall are connected preferably using welding or bonding, thereby ensuring that the pocket wall is connected securely and via a tight connection to the balloon wall. In one embodiment, the joint between the balloon wall and the pocket wall can also be used as a predetermined breaking point. In that case, the joint tears open when the balloon is dilated, thereby losing its seal integrity.

According to a preferred development of the invention, the predetermined breaking point is designed as a point or a line. Particularly preferably, a large number of predetermined breaking points is disposed in a balloon wall or a pocket wall in the region of the particular reservoir. The design of the predetermined breaking point that is selected also defines the discharge behavior of the pharmaceutically active substance and the pressure with which it is injected into the tissue. In particular, when a punctiform predetermined breaking point is used, a high discharge pressure and therefore a great penetration depth into the tissue can be achieved since the predetermined breaking point functions as a nozzle. A preferred density of punctiform predetermined breaking points, which are preferably disposed in the region of the reservoir, distributed at regular intervals over the particular wall, is 1 to 500 predetermined breaking points per cm². In one embodiment, a linear predetermined breaking point can be designed as a ring or a spiral i.e. encircling the cylindrical balloon in the shape of a ring or spiral. Linear predetermined breaking points are preferably separated by a lateral distance of 250 μm to 5 mm, particularly preferably by a distance of 500 μm to 3 mm.

Furthermore, it is preferable for the balloon wall and/or the outer wall and/or the pocket wall to have a smaller thickness in the region of the predetermined breaking point than in the remaining region of the particular wall, preferably a thickness of 5% to 75%, particularly preferably a thickness of 10% to 25% of the thickness of the particular wall in the rest of the region. Providing a thinner wall in the region of the predetermined breaking point than in the remaining region of the particular wall is a particularly simple way to implement a predetermined breaking point.

According to a further advantageous possibility for implementing a predetermined breaking point, the material of the balloon wall or the outer wall or the pocket wall is weakened in the region of the predetermined breaking point, wherein the weakening is created preferably using a laser, etching, material removal (e.g. cutting, perforation), the application of a joint, and/or material embrittlement. The material embrittlement can be created e.g. while the balloon is being manufactured by bending the material repeatedly and in a cyclic manner. The region of the brittle material is created at the pressure ridge or bending edge. Microcracks may also be present here.

Furthermore, a predetermined breaking point can be realized in that the balloon wall and/or the outer wall and/or the pocket wall are composed of a material in the region of the predetermined breaking point that differs from the material provided in the adjacent, remaining region of the particular wall. The material that is used in the region of the predetermined breaking point is preferably softer or more brittle than the material in the remaining region of the particular wall. The same polymer, for instance, that is also used for the pocket wall or the balloon wall, although in a longer-chained or shorter-chained form, can be used at the predetermined breaking point. The material of the predetermined breaking point is joined with the material of the pocket wall or the balloon wall during manufacture of the pocket wall or balloon wall e.g. via extrusion, injection, etc. Long-chained and short-chained polymers of the same class have other strengths and material properties. As an alternative, it is also possible to use two polymers having different strengths/brittleness, which are subsequently bonded or welded.

The material for the balloon wall or the pocket wall can be e.g. polyethylenes (PET), polycarbonates (PC), polyamides (PA), polyimides (PI), PEBAX, PA11, PA12 or PVC and their blends. Furthermore, these materials can be provided with a compounding/blending with the polymer class of silicones or, more generally, of the thermoplastic and/or elastomeric polymers.

The above-described object is also solved, with the aforementioned advantages, by a system for applying an intraluminal endoprosthesis, in which case the intraluminal endoprosthesis is disposed on the outside of the balloon, preferably being crimped thereto. In this state, the balloon is not yet dilated. Dilating the balloon of the catheter simultaneously causes the intraluminal endoprosthesis disposed on the outside of the balloon to become permanently expanded; after the balloon is deflated, the intraluminal endoprosthesis remains in the particular body cavity.

According to a particularly preferred embodiment, the intraluminal endoprosthesis is designed as a biodegradable stent, preferably as an AMS (absorbable metal stent) or an absorbable polymer stent. Stents of that type contain a biodegradable metal as the main component, preferably magnesium, iron, zinc, tungsten, and/or an alloy of the aforementioned metals. Polymer stents are composed of degradable polymers.

The term “biodegradation” is understood to mean hydrolytic, enzymatic, and other metabolic degradative processes in the living organism, which are caused primarily by the bodily fluids that come in contact with the endoprothesis and result in a gradual disintegration of at least large portions of the endoprosthesis. The term “biocorrosion” is often used as a synonym for the term “biodegradation”. The term “bioresorption” includes the subsequent resorption of the degradative products by the living organism. Biodegradable materials can be composed of degradable polymers or the aforementioned metals.

The above-described object is furthermore solved by a method, in which a catheter body comprising an inner shaft and possibly an outer shaft is provided, then a balloon inner wall and a balloon outer wall are manufactured, the balloon inner wall and the balloon outer wall are connected to one another after a pharmaceutically active substance is placed between the two walls, and the balloon is connected to the inner shaft and possibly the outer shaft. At least one predetermined breaking point is formed in the outer wall of the balloon, either while the balloon is being manufactured, or it is installed subsequently.

The reservoir geometry of the dual lumen balloon manufactured using the method described above can be described, in the simplest case, as a hollow cylinder having a jacket thickness M, wherein M includes the distance between the outer surface of the balloon inner wall and the inner surface of the balloon outer wall in the balloon central part. Jacket thickness M is e.g. between 50 μm and 1 mm, in particular between 100 μm and 750 μm. In such an embodiment, punctiform predetermined breaking points, for example, are also distributed at regular intervals over the balloon outer wall.

The above-described object is also solved using a method in which a catheter body comprising an inner shaft and possibly an outer shaft is provided, then a balloon wall is manufactured, and a pocket wall for forming a pocket as a reservoir for at least one pharmaceutically active substance is now situated on the inner side or the outer side of the balloon wall, wherein, after the at least one pharmaceutically active substance is placed between the balloon wall and a pocket wall, the pocket wall is connected to the balloon wall, and then the balloon is connected to the inner shaft and possibly the outer shaft of the catheter body, wherein at least one predetermined breaking point is formed in the balloon wall or the pocket wall.

The methods described above are simple methods for manufacturing an advantageous catheter comprising a reservoir, the outwardly sealing wall of which comprises at least one predetermined breaking point.

As an alternative to the above-described methods, the at least one pharmaceutically active substance can be added via diffusion, for instance, into the particular reservoir subsequently, i.e. after the balloon has been manufactured with an (empty) reservoir.

Preferably, the balloon inner wall and the balloon outer wall and/or the balloon wall and the pocket wall are connected to one another using a joining process, preferably welding or bonding.

The at least one predetermined breaking point can be manufactured preferably by weakening the material of the balloon wall and/or the pocket wall, the weakening being created by using a laser, etching, material removal, applying a joint, and/or material embrittlement.

Preferably, reservoir pockets are prefabricated with the pocket wall onto which the pharmaceutically active substance is to be applied, and possibly with the at least one predetermined breaking point, and, once the balloon wall is completed, are bonded only to the inside or the outside of the balloon wall, or are connected to the balloon wall via welding.

A catheter that is manufactured according to such methods results in simple handling of a balloon provided with a drug, without risking the safety of the user. By placing the pharmaceutically active substance in a reservoir, a drug coating is prevented from becoming scraped off during surgery and contaminating the surroundings. Furthermore, by placing the pharmaceutically active substance in a reservoir which is initially closed, it is possible to use a large number of pharmaceutically active substances which are not available for a coating. In addition, the pharmaceutically active substance can be dosed more accurately and placed more exactly in the body cavity to be treated.

The object described above is furthermore solved by a simple and cost-effective method for manufacturing a system in which the catheter is manufactured according to one of the possibilities described above, and the intraluminal endoprosthesis is then fastened to the outside of the preferably folded balloon, preferably being crimped thereto, in such a manner that the former at least partially encloses the latter. Particularly preferably and in a very simple manner, the intraluminal endoprosthesis is disposed to the balloon using crimping.

Further objectives, features, advantages, and possible applications of the invention result from the following description of embodiments, with reference to the figures. All of the features that are described and/or graphically depicted are the subject of the present invention, either alone or in any combination, independently of their wording in the claims or their back-references.

### DESCRIPTION OF THE DRAWINGS

The drawings show, schematically:

FIG. 1 a longitudinal sectional view of a first balloon for a catheter, according to the invention, in the non-dilated state,

FIG. 2 a view of the balloon according to FIG. 1, from the side,

FIG. 3 a longitudinal sectional view of a second balloon for a catheter, according to the invention, in the non-dilated state,

FIG. 4 a view of the balloon according to FIG. 2, from the side,

FIG. 5 a longitudinal sectional view of a first embodiment of a catheter according to the invention,

FIG. 6 a longitudinal sectional view of a second embodiment of a catheter according to the invention,

FIG. 7 shows a first embodiment of a system according to the invention, in a view from the side, and

FIG. 8 shows a second embodiment of a system according to the invention, in a view from the side.

### DETAILED DESCRIPTION

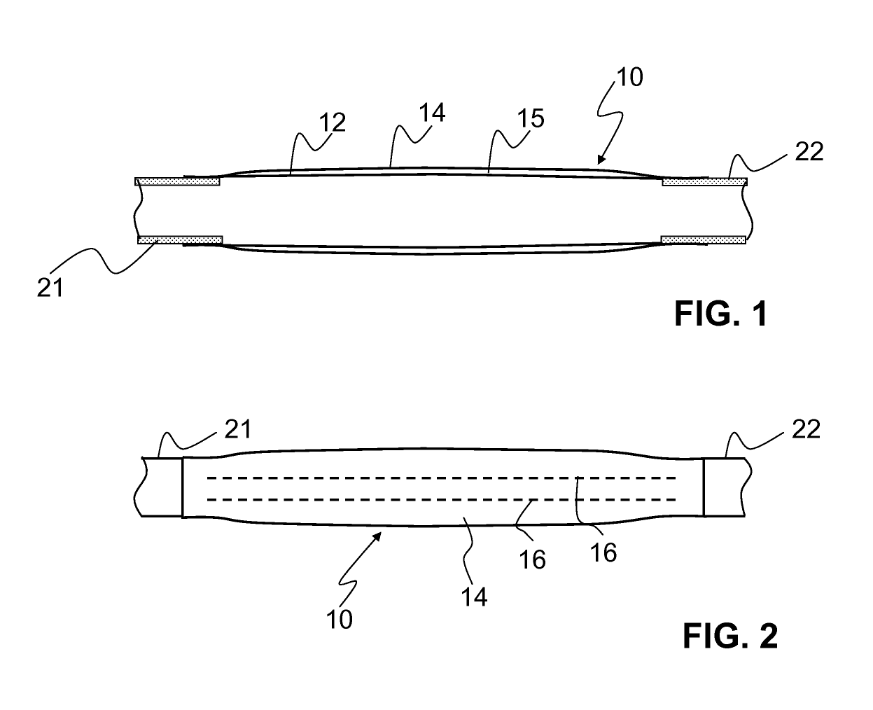

FIG. 1 shows the initial shape of a balloon 10 for a catheter according to the invention, which is disposed via the distal end thereof on a first shaft 21 and via the proximal end thereof on a second shaft 22. Balloon 10 comprises an inner wall 12 and an outer wall 14 which are connected to one another at the distal and proximal ends. A reservoir 15 is formed between inner wall 12 and outer wall 14, in which the at least one (not depicted) pharmaceutically active substance is disposed. The materials used for inner wall 12 and outer wall 14 can be any of the common balloon material such as polyethylenes (PET), polycarbonates (PC), polyamides (PA), polyimides (PI), PEBAX, PA11, PA12 or PVC and their blends.

Linear predetermined breaking points 16, which have been manufactured e.g. using a laser, are formed in outer wall 14 of balloon 10. The thermal energy applied using the laser weakened the material in the region of lines 16. The weakening of the material has an effect similar to that of material removal. When dilation pressure is applied, the pressure seeks out the least resistance. Thus, the reservoir will preferably tear open at these points. The material of outer wall 14 of balloon 10 is weakened in the region of linear predetermined breaking point 16. When balloon 10 is dilated, predetermined breaking points 16 would tear open, thereby releasing the pharmaceutically active substance(s) disposed in reservoir 15. Predetermined breaking points 16 are indicated in FIG. 2 using dashed lines.

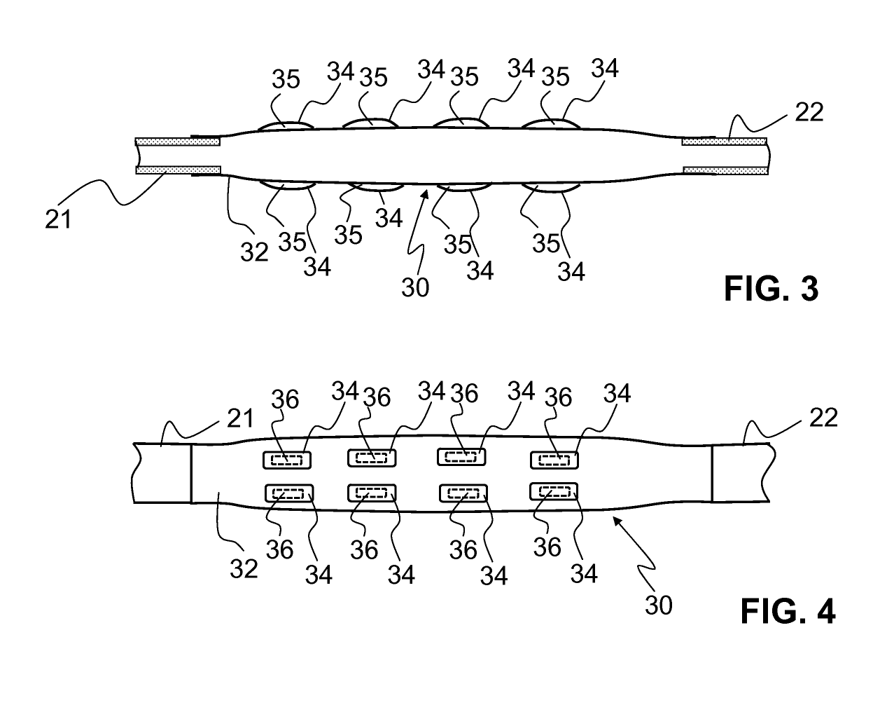

FIG. 3 shows a second embodiment of a balloon 30 for a catheter according to the invention. Similarly to balloon 10 depicted in FIG. 1, balloon 30 is disposed via the distal end thereof on a first shaft 21 and via the proximal end thereof on a second shaft 22. Balloon 30 includes a balloon wall 32 having pockets disposed on the outer side. Each pocket has a pocket outer wall 34 which, in combination with balloon wall 32, forms particular reservoir 35 for the pharmaceutically active substance. Pocket outer wall 34 is connected to balloon wall 32 using welding or bonding. Welding processes are preferably carried out using lasers. In that case, the surface of the parts to be connected are melted without their material properties being changed, and balloon wall 32 and pocket outer wall 34 are then connected. The bonding is carried out using suitable adhesive means and methods. For cold joining, adhesives of the epoxide or acrylic material classes can be used. The two parts are pressed together to be joined. For thermal joining processes, any thermoplastic polymer material can be used, namely to wet the particular balloon wall material or pocket wall material. First, the joint is wetted with adhesive and is then molded, with the application of heat (e.g. using an IR radiator).

At least one pharmaceutically active substance (not depicted), possibly in microcapsules, is provided in each reservoir 35.

FIG. 4 shows that each pocket having outer wall 34 is provided with punctiform predetermined breaking points in oval region 36 of pocket outer wall 34, which is bordered by a dashed line. In region 36, the punctiform predetermined breaking points are distributed at regular intervals in a density of 1 to 500 predetermined breaking points per cm². The punctiform predetermined breaking points have the properties described in conjunction with linear predetermined breaking points 16. In particular, the predetermined breaking points do not extend through the material entirely. The predetermined breaking points can be applied using mechanical, chemical, or thermal methods. The balloon is manufactured up to this step using conventionally known methods. After the predetermined breaking points are opened in region 36 when balloon 30 is dilated, the pharmaceutically active substance can pass through them and are injected into the surrounding tissue (not depicted) of the body cavity to be treated.

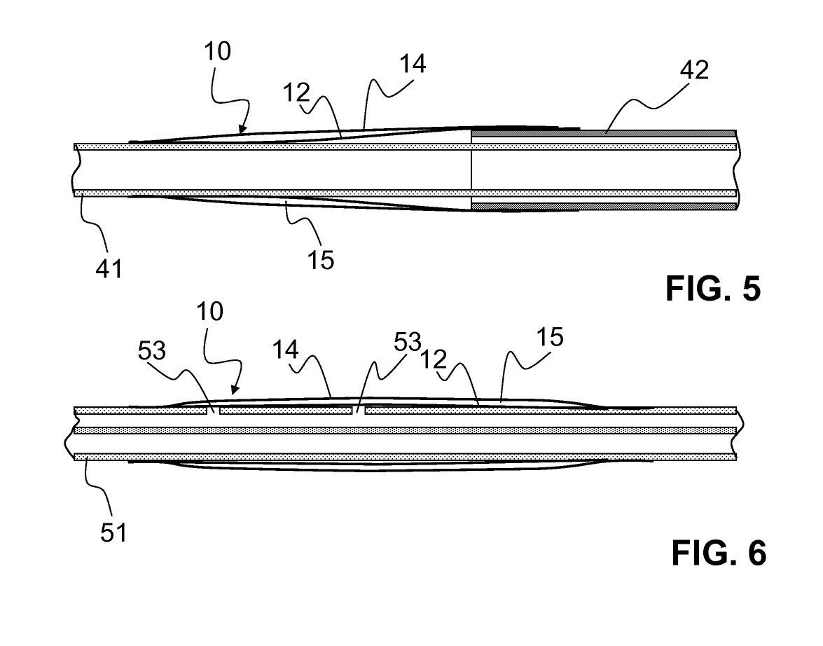

In the first embodiment, which is depicted in FIG. 5, the catheter according to the invention comprises an inner shaft 41, to which the distal balloon neck of balloon 10 is connected. The proximal balloon neck of balloon 10 is connected to an outer shaft 42 of the catheter, wherein outer shaft 42 is disposed concentrically on inner shaft 41 and encloses it. The hollow-cylindrical cavity formed between outer shaft 42 and inner shaft 41 makes it possible for balloon 10 to be inflated, thereby allow it to dilate. As an alternative to the embodiment depicted in FIG. 5, balloon 30 can also be disposed on inner shaft 41 and inner shaft 42, of course, as shown in FIGS. 3 and 4.

FIG. 6 shows a second embodiment of a catheter according to the invention, which comprises a dual lumen inner shaft 51. In this embodiment, the distal balloon neck and the proximal balloon neck of balloon 10 are connected to inner shaft 51. A lumen of dual lumen inner shaft 51 comprises openings 53, using which balloon 10 can be inflated. Balloon 30 depicted in FIGS. 3 and 4 can likewise be disposed on such a dual lumen inner shaft 51.

To manufacture a system according to the invention, an intraluminal endoprosthesis, e.g. a stent 60, is now installed on balloon 10 or balloon 30 once the particular catheter has been completed. Preferably, such a stent 60 is crimped onto a balloon 10 or 30. In the handling process, stent 60 is expanded simultaneously with the dilation of balloon 10 or balloon 30 and the body cavity. After particular balloon 10, 30 is deflated, stent 60 remains in the body cavity and supports it.

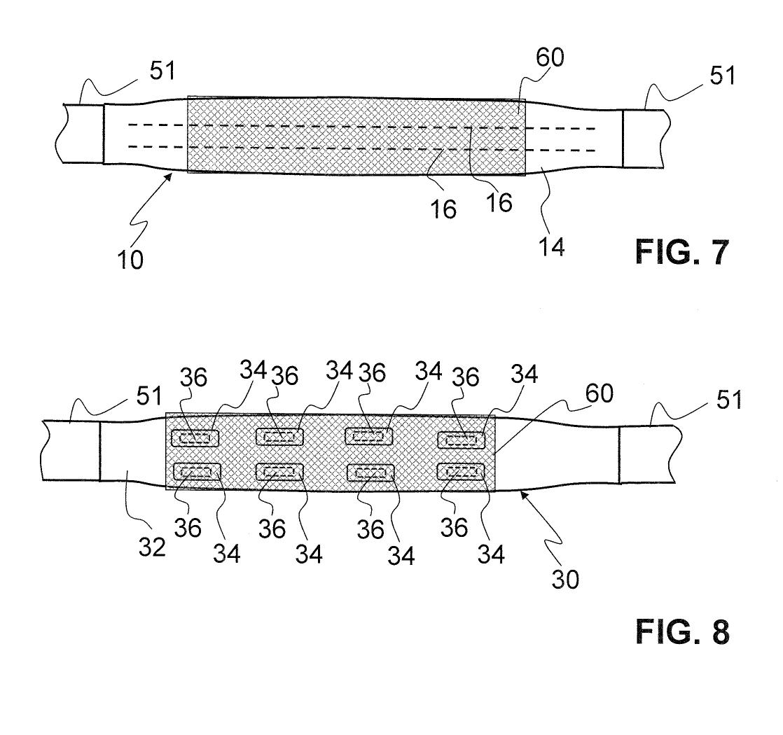

FIG. 7 shows the catheter depicted in FIG. 6 with balloon 10 described with reference to FIGS. 1 and 2, onto which stent 60 has been crimped. Thus, the catheter depicted in FIG. 6 is shown in FIG. 8 with balloon 30 and stent 60 shown in FIGS. 3 and 4.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

### LIST OF REFERENCE CHARACTERS

10 Balloon
12 Inner wall of balloon 10
14 Outer wall of balloon 10
15 Reservoir
16 Linear predetermined breaking point
21 First shaft
22 Second shaft
30 Balloon
32 Balloon wall
34 Pocket outer wall
35 Reservoir
36 Region in which punctiform predetermined breaking points are disposed
41 Inner shaft
42 Outer shaft
51 Inner shaft
53 Opening
60 Stent

What is claimed is:

1. A catheter comprising a balloon and at least one pharmaceutically active substance, characterized in that the pharmaceutically active substance is disposed in a reservoir inside the balloon or on the balloon, wherein a wall that closes the reservoir toward the outside comprises at least one predetermined breaking point.

2. The catheter according to claim 1, characterized in that the balloon comprises an inner wall and an outer wall, and the reservoir is formed between the inner wall and the outer wall.

3. The catheter according to claim 1, characterized in that a pocket is formed as the reservoir on an inner side or an outer side of a balloon wall using a pocket wall, wherein the pocket wall is connected along its entire circumference to the balloon wall using a joining process, optionally welding or bonding.

4. The catheter according to claim 1, characterized in that the predetermined breaking point is designed as a point or a line.

5. The catheter according to claim 1, characterized in that a balloon wall and/or an outer wall and/or a pocket wall have a smaller thickness in a region of the predetermined breaking point than in a remaining region of the particular wall, optionally a thickness of 5% to 75%, and optionally a thickness of 10% to 25% of the thickness of the particular wall in a rest of the region.

6. The catheter according to claim 1, characterized in that the material of a balloon wall and/or an outer wall and/or a pocket wall is weakened in a region of the predetermined breaking point, wherein the weakening is optionally created using a laser, etching, material removal, the application of a joint, and/or material embrittlement.

7. The catheter according to claim 1, characterized in that a balloon wall and/or an outer wall and/or a pocket wall is composed of a material in a region of the predetermined breaking point that differs from material provided in an adjacent, remaining region of the particular wall.

8. The catheter according to claim 1, characterized in that a density of punctiform predetermined breaking points which are optionally disposed in a region of the reservoir, distributed at regular intervals over a balloon wall and/or an outer wall and/or a pocket wall, is 1 to 500 predetermined breaking points per cm².

9. The catheter according to claim 1, characterized in that the predetermined breaking points are linear and separated by a lateral distance of 250 μm to 5 mm, optionally 500 μm to 3 mm.

10. A system for applying an intraluminal endoprosthesis, optionally a stent, in a body cavity, comprising the intraluminal endoprosthesis and a catheter according to claim 1, wherein the intraluminal endoprosthesis is disposed on the outside of the balloon, optionally being crimped thereto.

11. The system according to claim 10, characterized in that the intraluminal endoprothesis is designed as a biodegradable stent.

12. A method for manufacturing a catheter, in particular according to claim 1, characterized by the steps of:
providing a catheter body comprising an inner shaft and possibly an outer shaft,
manufacturing a balloon inner wall and a balloon outer wall,
placing a pharmaceutically active substance between the two walls,
connecting the balloon inner wall and the balloon outer wall to one another,
connecting the balloon to the inner shaft and possibly the outer shaft, and
forming at least one predetermined breaking point in the outer wall of the balloon.

13. The method according to claim 12, characterized in that the balloon inner wall and the balloon outer wall and/or the balloon wall and the pocket wall are connected to one another using a joining process, optionally welding or bonding.

14. The method according to claim 12, characterized in that the predetermined breaking point is designed as a point or a line.

15. The method according to claim 12, characterized in that the predetermined breaking point is created by weakening the material of the outer wall and/or the balloon wall and/or the pocket wall, preferably using a laser, etching, material removal, the application of a joint, and/or material embrittlement.

16. The method according to claim 12, characterized in that the balloon wall and/or the outer wall and/or the pocket wall have a smaller thickness in the region of the predetermined breaking point than in the remaining region of the particular wall, optionally a thickness of 5% to 75%, and optionally a thickness of 10% to 25% of the thickness of the particular wall in a rest of the region.

17. A method for manufacturing a catheter, in particular according to claim 1, characterized by the steps of:
providing a catheter body comprising an inner shaft and possibly an outer shaft,
manufacturing a balloon wall and a pocket wall to form a pocket as a reservoir for at least one pharmaceutically active substance situated on an inner side or outer side of the balloon wall,
placing at least one pharmaceutically active substance between the balloon wall and a pocket wall,
connecting the pocket wall to the balloon wall,
connecting the balloon to the inner shaft and possibly the outer shaft of the catheter body, wherein at least one predetermined breaking point is formed in the balloon wall or the pocket wall.

18. A method for manufacturing a system for applying an intraluminal endoprosthesis, optionally a stent, in a body cavity, comprising the intraluminal endoprosthesis and a catheter, wherein the catheter is manufactured according to a method described in claim 12, and then the intraluminal endoprosthesis is fastened to the outside of the balloon, which is optionally folded and optionally crimped thereto, in such a manner that the intraluminal endoprosthesis at least partially encloses the balloon.
